(19) United States
(12) Patent Application Publication
(10) Pub. No.: US 2014/0106012 A1
(43) Pub. Date: Apr. 17, 2014

(54) COMPOSITIONS AND METHODS USEFUL FOR TREATING PEDICULOSIS

(71) Applicant: PHARMAYEDA LTD., Kfar Netter (IL)

(72) Inventors: Orna LEVIN, Kfar Netter (IL); Yochanan Forman, Kibbutz Maabarot (IL)

(21) Appl. No.: 14/137,715

(22) Filed: Dec. 20, 2013

Related U.S. Application Data
(63) Continuation-in-part of application No. PCT/IB2012/053109, filed on Jun. 20, 2012.
(60) Provisional application No. 61/499,450, filed on Jun. 21, 2011.
(60) Provisional application No. 61/745,694, filed on Dec. 24, 2012.

(30) Foreign Application Priority Data
Jun. 20, 2012 (IB) PCT/IB2012/053109

(51) Int. Cl. A61K 36/53; A61K 45/06; A61K 36/23; A61K 31/695; A61K 31/23

(52) U.S. Cl. CPC A61K 36/53; A61K 31/695; A61K 31/23; A61K 36/23; A61K 45/06
USPC 424/739; 424/725

(57) ABSTRACT

Disclosed are compositions and methods that in some embodiments are useful in the treatment of pediculosis.

### RELATED APPLICATION

The present application is a Continuation-in-Part of PCT/IB2012/053109 having an International Filing Date of Jun. 20, 2012 that was published on Dec. 27, 2012 as WO2012/176131 that takes priority from U.S. Provisional Patent Application No. 61/499,450 filed Jun. 21, 2011 both which are incorporated by reference as if fully set-forth herein. The present application also takes priority from U.S. Provisional Patent Applications No. 61/745,694 filed Dec. 24, 2012, which is incorporated by reference as if fully set-forth herein.

### FIELD AND BACKGROUND OF THE INVENTION

The invention, in some embodiments, relates to the field of pest control, and more specifically, but not exclusively, to methods and compositions for treating infestations by insects, especially infestations by lice (pediculosis).

Lice belong to the group of external parasites living on warm-blooded animals. An infestation of lice is called pediculosis. In humans there are three different varieties of pediculosis caused by different types of lice: (1) Pediculosis pubis in the pubic area, caused by Pithirus pubis; (2) Pediculosis corporis on the trunk, caused by Pediculus humanus humanus; and (3) Pediculosis capitis on the head, caused by Pediculus humanis capitis. Pediculosis affects millions of humans world-wide.

A variety of treatments for lice infections are known in the art, but none has succeeded in eradicating this condition. Epidemics of pediculosis appear to be cyclical in nature.

Essential oils are well known in the art for the treatment of medical problems. Essential oils in connection with the present invention are those essential oils embraced by the definition in Hackh's Chemical Dictionary, 4^{th} Edition, page 248.

Although certain essential oils, such as rosemary oil, have been suggested for use in treating pediculosis, essential oils have a number of drawbacks. First, essential oils are rather expensive. Moreover, the traditional way to use essential oils, by direct application of the pure oil to the skin, frequently causes skin irritation accompanied by a burning sensation and erythema. Diluting the essential oils in a fixed oil such as olive oil may reduce such side-effects but also reduces the efficacy of the essential oil. Dissolving an essential oil in an alcohol, e.g., ethanol, often allows preservation of anti-insect properties but generally increases undesirable side-effects, e.g., the burning sensation on the skin and erythema.

PCT Application WO 01/26471 of one of the Inventors teaches a composition for the pediculosis comprising at least one essential oil, preferably anise oil; at least one alcohol; and at least one fixed oil and/or one or more emollient ester of fatty acid derived from vegetable oils. An embodiment of the teachings thereof is commercially available in the United States as “Hairclean 1-2-3”® or Scram®, and in Europe as Paranix®. Typically, the composition is applied to the hair and maintained in contact therewith for at least 15 minutes followed by use of a fine-tooth comb to remove nits and lice. Tests show that such treatment eliminates at least 98% of mature lice and 52% nits, the eggs of lice that are attached to the hair with a waterproof adhesive. Such a composition has a combination of essential oils (rosemary and/or anise oils) and a relatively high concentration of alcohols (ethanol or isopropyl alcohol) which can produce adverse topical reactions, such as contact dermatitis in sensitive subjects.

Another type of composition for treating pediculosis includes a combination of silicon oil and isopropyl myristate, for example commercially available as Resultz® (50% cyclomethicone and 50% isopropyl myristate, from Nycomed Canada Inc., Oakville, Ontario, Canada). Such compositions kill adult lice through two modes of action. The isopropyl myristate dissolves the waxy coating of the louse exoskeleton, leading to dehydration of the louse, while the silicone oil, acting as a lubricant, makes it difficult for weakened lice to grasp the hair such that the lice are easy to comb out from the hair. Additionally, the silicone oil may have some lice-smothering effect. For use, the composition is carefully applied to the scalp and the entire length of the hair shafts, maintained in contact therewith for at least 10 minutes followed by washing, rinsing and combing with a fine-tooth comb to remove dead lice. Even after washing and rinsing, the composition leaves an unpleasant residue that has the advantage of making combing easier.

Additional background can be found in PCT publications WO 2009/144712 and WO 2001/1026471; Israel Patent IL 98828; US patent application US 2008/0193387 and in Burgess I. F. “Current treatments for pediculosis capitis” in Current Opinion and Infectious Diseases 2009; 22” 131-136.

It is desirable to have a composition for the treatment of lice that has some advantage over known treatments.

### SUMMARY OF THE INVENTION

The invention, in some embodiments thereof, relates to a composition useful for the treatment of pediculosis and, more particularly, but not exclusively, to a composition useful for the treatment of pediculosis comprising an essential oil.

According to some embodiments of the invention, there is provided a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and an emollient ester of fatty acid derived from vegetable oils.

In some embodiments, the essential oil is selected from the group consisting of anise oil and rosemary oil.

According to some embodiments, there is provided a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils, for use in treating pediculosis.

According to some embodiments, there is provided the use of a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils, in the manufacture of a medicament for the treatment of pediculosis.

According to some embodiments, there is provided a method for the treatment of pediculosis, comprising topically administering to a subject in need thereof a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils.

In some embodiments of the method of the present invention, the composition remains in contact with an area of skin of said subject for a time period of from about 1 to about 15 minutes, such as, for example, about 5 minutes. In some embodiments, the subject is a human. In some embodiments, the subject is a non-human animal.

In some embodiments of the method or use of the present invention, the pediculosis is selected from the group consisting of pediculosis capitis and pediculosis humanus humanus.

According to some embodiments, there is provided a method for the preparation of a composition useful in the treatment of pediculosis comprising mixing together an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils.

In some embodiments, the essential oil is selected from the group consisting of anise oil and rosemary oil.

In some embodiments, the solvent comprises a combination of isopropyl alcohol and isopropyl myristate.

In some embodiments, the silicone oil comprises dimethicone. In some embodiments, dimethicone is present at a concentration of from about 5% to about 20% volume per volume.

In some embodiments, the emollient ester of fatty acid comprises caprylic/capric triglyceride. In some embodiments, the caprylic/capric triglyceride is present at a concentration of from about 5% to about 25% volume per volume.

In some embodiments, the composition comprises rosemary oil or anise oil, isopropyl alcohol, isopropyl myristate, caprylic/capric triglyceride and a silicone oil (preferably dimethicone). In some embodiments, the composition consists essentially of rosemary oil or anise oil, isopropyl alcohol, isopropyl myristate, caprylic/capric triglyceride and a silicone oil (preferably dimethicone). In some embodiments, the composition consists of rosemary oil or anise oil, isopropyl alcohol, isopropyl myristate, caprylic/capric triglyceride and a silicone oil (preferably dimethicone).

In some embodiments, the essential oil is present at a concentration of from about 1% to about 20% volume per volume.

In some embodiments, the isopropyl alcohol is present at a concentration of from about 10% to about 30% volume per volume.

In some embodiments, the isopropyl myristate is present at a concentration of from about 30% to about 75% volume per volume, from about 30% to about 65% volume per volume and in some embodiments from about 30% to about 50% volume per volume.

In some embodiments, the composition comprises one of rosemary oil and anise oil as sole essential oil.

In some embodiments, the composition is formulated for topical administration. According to some aspects of the present invention, there is provided the use of the composition of any of the embodiments described herein, for killing of nits.

Unless otherwise defined, all technical and scientific terms used herein have the same meaning as commonly understood by one of ordinary skill in the art to which this invention pertains. In case of conflict, the patent specification, including definitions, will control.

As used herein, the term “treating” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the term “pharmaceutically acceptable” means approved by a regulatory agency of the US Federal or a US state government or listed in the U.S. Pharmacopeia or other generally recognized pharmacopeia for use in animals, and more particularly in humans. Herein, the phrase “pharmaceutically acceptable carrier” refers to an approved carrier or a diluent that does not cause significant irritation to an organism and does not abrogate the biological activity and properties of the administered conjugate.

As used herein, the term “carrier” refers to a diluent, adjuvant, excipient, or vehicle with which the therapeutic is administered.

Herein the term “excipient” refers to an inert substance added to a pharmaceutical composition to further facilitate processes and administration of the active pharmaceutical ingredients.

As used herein, the indefinite articles “a” and “an” mean “at least one” or “one or more” unless the context clearly dictates otherwise.

As used herein, when a numerical value is preceded by the term “about” the term “about” is intended to indicate +/−10%.

### DESCRIPTION OF SPECIFIC EMBODIMENTS OF THE INVENTION

The invention, in some embodiments thereof, relates to a composition useful for the treatment of pediculosis and, more particularly, but not exclusively, to a composition useful for the treatment of pediculosis comprising an essential oil.

In some embodiments, the essential oil is selected from the group consisting of anise oil, rosemary oil, calendula oil, tea tree oil, sassafras oil, quassia oil, cinnamon oil (including oil of leaf, bark, fruit, or twig of the cinnamon plant, or combinations thereof), clove oil (including oil of the flower, bud or leaf, or combinations thereof), eucalyptus oil (including citriodora, radiata, smithii, staigeriana and globulus, or combinations thereof), lavender oil (including angustifolia and latifolia), peppermint oil, or combinations thereof.

In some embodiments, the essential oil is selected from the group consisting of anise oil and rosemary oil.

The principles, uses and implementations of the teachings herein may be better understood with reference to the accompanying description. Upon perusal of the description, one skilled in the art is able to implement the invention without undue effort or experimentation.

Before explaining at least one embodiment in detail, it is to be understood that the invention is not necessarily limited in its application to the details of the embodiments set forth herein. The invention is capable of other embodiments or of being practiced or carried out in various ways. The phraseology and terminology employed herein are for descriptive purpose and should not be regarded as limiting.

As mentioned above, PCT Application WO 01/26471 (an embodiment of which is commercially available) discloses compositions for the treatment of pediculosis which are applied to the scalp of a subject and are generally kept in contact with the hair and scalp for a period of at least 15 minutes for maximal efficacy. Such a time period can be seen as inconveniently long, especially when treating young children who may have limited patience and must be continually prevented from touching the head once the composition is applied. Use of such compositions has resulted in 100% mortality of adult lice but insufficient nit mortality. As a result, repeated application is required after 7-10 days when a new batch of lice nymphs hatch from surviving nits. In addition, the compositions described in the PCT application either comprise a combination of rosemary oil and anise oil (both which are expensive) and/or use a relatively high concentrations of ethanol and/or isopropyl alcohol, which can cause contact dermatitis and other adverse topical reactions in particularly sensitive subjects.

The present invention provides a composition for the treatment of lice infestation. Some embodiments of the composition comprises a single essential oil and not a combination of essential oils. Some embodiments of the composition have a comparatively low alcohol content, reducing potential undesirable side-effects. Some embodiments of the composition are fast-acting, in some embodiments requiring even less than 15 minutes contact with the hair and scalp for maximal efficacy. Some embodiments of the composition are unexpectedly effective in killing nits, even close to 100% nit mortality 10 days after application. Accordingly, some embodiments of a composition as described herein have been experimentally proven to be at least as effective and/or to have advantages in the treatment of lice infestation compared to the compositions disclosed in WO 01/26471.

Although not wishing to be held to any one theory, it is currently believed that the combination of ingredients making up some embodiments of the composition described herein act synergistically, in some embodiments allowing a relatively low amount of some individual components to be used, thereby reducing the probability of adverse side effects, while the combined effects of the components provide a composition which is demonstrated to be effective in the treatment of lice infestation.

In some embodiments, the minimal recommended application time for maximal efficacy is equal or shorter than that required with prior art compositions.

In some embodiments, the composition disclosed herein is effective against nits and hatched lice (nymphs and adults) when topically applied to a subject infested with lice, such that, all things being equal, hatching of nits and recurrence of the infestation is less likely to occur.

### Composition and Preparation of Composition

According to some embodiments of the invention, there is provided a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils. In some embodiments, such a composition is useful in the treatment of pediculosis. In some embodiments, such a composition is useful in the manufacture of a medicament for the treatment of pediculosis.

According to some embodiments of the invention, there is provided a composition comprising: an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof (preferably anise oil, rosemary oil or a combination of anise oil and rosemary oil); as a solvent, a combination of between about 10% to about 30% (v/v) isopropyl alcohol and between about 30% to about 75% (v/v) isopropyl myristate; between about 5% and about 20% (v/v) of a silicone oil (preferably dimethicone); and between about 5% and about 25% (v/v) of at least one emollient ester of fatty acid derived from vegetable oils (preferably capric/caprylic triglyceride). In some embodiments, such a composition is useful in the treatment of pediculosis. In some embodiments, such a composition is useful in the manufacture of a medicament for the treatment of pediculosis.

According to some embodiments of the invention, there is provided a method for the treatment of pediculosis comprising: topically administering to a subject in need thereof a composition comprising an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils.

According to some embodiments of the invention, there is provided a method for the treatment of pediculosis comprising topically administering to a subject in need thereof a composition comprising: an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof (preferably anise oil, rosemary oil or a combination of anise oil and rosemary oil); as a solvent, a combination of between about 10% to about 30% (v/v) isopropyl alcohol and between about 30% to about 75% (v/v) isopropyl myristate; between about 5% and about 20% (v/v) of a silicone oil (preferably dimethicone); and between about 5% and about 25% (v/v) of at least one emollient ester of fatty acid derived from vegetable oils (preferably capric/caprylic triglyceride).

According to some embodiments of the invention, there is provided a method for the preparation of a composition useful in the treatment of pediculosis comprising: combining an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof; at least one solvent selected from the group consisting of isopropyl alcohol and isopropyl myristate; a silicone oil; and at least one emollient ester of fatty acid derived from vegetable oils.

According to some embodiments of the invention, there is provided a method for the preparation of a composition useful in the treatment of pediculosis comprising combining: an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof (preferably anise oil, rosemary oil or a combination of anise oil and rosemary oil); as a solvent, a combination of between about 10% to about 30% (v/v) isopropyl alcohol and between about 30% to about 75% (v/v) isopropyl myristate; between about 5% and about 20% (v/v) of a silicone oil (preferably dimethicone); and between about 5% and about 25% (v/v) of at least one emollient ester of fatty acid derived from vegetable oils (preferably capric/caprylic triglyceride).

In some embodiments, the essential oil comprises a combination of anise oil and rosemary oil. That said, in preferred embodiments, either anise oil or rosemary oil comprises the sole essential oil.

In some embodiments, the at least one solvent comprises a mixture of isopropyl alcohol and isopropyl myristate.

In some embodiments, the silicone oil is selected from the group consisting of a polyalkyl siloxane, a polyaryl siloxane, a polyalkylaryl siloxane or a polyether siloxane copolymer, and combinations thereof. In some embodiments, the polyalkyl siloxane comprises polymethyl siloxane (dimethicone).

In some embodiments, the emollient ester of fatty acid is selected from the group consisting of medium chain triglycerides (MCT), caprylic/capric triglyceride; propylene glycol dicaprylate-dicaprate; and isopropyl palmitate. In some embodiments, the ester comprises caprylic/capric triglyceride.

The composition described herein may be manufactured using processes well known in the art, e.g., by means of conventional mixing, dissolving, emulsifying etc. Further techniques for formulation and administration of active ingredients may be found in “Remington's Pharmaceutical Sciences,” Mack Publishing Co., Easton, Pa., latest edition, which is incorporated by reference as if fully set forth herein.

In some embodiments, a composition as described herein further comprises a suitable carrier to facilitate administration.

In some embodiments, a composition as described herein further comprises additional excipients and auxiliaries, known to those skilled in the art, to facilitate processing of the active ingredients into preparations which, can be used pharmaceutically. Non-limiting examples of such excipients include anti-oxidants, gelling agents, thickening agents, stabilizers, and the like.

A composition as described herein may be used for treating any subject suffering from pediculosis, either a human or a non-human animal. A composition as described herein may be used in any suitable manner, but is preferably applied topically to the subject, most preferably to the skin and/or hair and/or pelt and/or fur of the subject. In some embodiments, the composition is used in a manner analogous to the use of “Hairclean 1-2-3”® or Resultz®.

For topical administration, a composition as described herein is provided in any suitable form which enables the composition to remain in contact with the skin and/or hair for a desired time period. Non-limiting examples of suitable forms include a liquid, a gel, a cream, an ointment, a paste, a lotion, a milk, a suspension, an aerosol, a spray, a foam, or a serum.

### Use of a Composition

In some embodiments, a composition as described herein is applied (e.g., by massaging into the hair) topically for a period of time up to about 15 minutes, up to about 10 minutes, and even up to about 5 minutes. In some embodiments, the composition is applied for about 5 minutes.

After the period of time, the hair is combed with a fine-tooth comb to remove lice and/or nits.

In some embodiments, after the composition has been left in contact with the skin and/or hair of the subject, the composition is removed either by rinsing with water or by washing with normal shampoo followed by rinsing.

### Rosemary Oil (CAS Nr. 8000-25-7)

Some embodiments of a composition as described herein comprise rosemary oil as an essential oil. Some preferred embodiments comprise rosemary oil as a sole essential oil. Some embodiments comprise rosemary oil together with at least one additional essential oil.

Rosemary is a woody, perennial herb with fragrant, evergreen, needle-like leaves, which is native to the Meditteranean region. Rosemary essential oil, which is extracted mainly from the leaves of the plant by steam distillation, is considered to stimulate hair growth, boost mental activity, relieve respiratory problems, reduce pain, and treat indigestion, flatulence and stomach cramps. Rosemary oil is used extensively for hair care in shampoos and lotions. Regular use of rosemary oil helps in stimulating follicles, as a result of which, hair grow longer and stronger. It is also believed that rosemary oil slows down premature hair loss and graying of hair. Rosemary essential oil is also beneficial for dry and flaky scalps. Regular massage of scalp with rosemary oil nourishes the scalp and removes dandruff. Further, it is often mixed with tea tree oil and basil oil to treat scalp problems. In PCT patent publication WO 01/26471, an antilice composition is disclosed having between about 3% and 70% rosemary oil.

In some embodiments, the composition comprises between about 1% and about 20%, in some embodiments between about 1% and about 15%, and in some embodiments even between about 5% and about 15% rosemary oil (v/v).

### Anise Oil (CAS Nr. 8007-70-3, Also 84775-42-8)

Some embodiments of a composition as described herein comprise anise oil as an essential oil. Some preferred embodiments comprise anise oil as a sole essential oil. Some embodiments comprise anise oil together with at least one additional essential oil.

Anise (Pimeinella anisum) is a herbaceous, annual flowering plant in the family Apiaceae, native to the eastern Mediterranean region and Southwest Asia. The essential oil, obtained by steam distillation of the seeds, mainly comprises anethole, a phytoestrogen. Anise oil is used in aromatherapy for bronchitis, colds and flu, for treatment of flautulence, impotence, menstrual pains, migraines and colic. In PCT patent publication WO 01/26471, an antilice composition is disclosed having between about 5% and 34% anise oil.

In some embodiments, the composition comprises between about 1% and about 20%, in some embodiments between about 1% and about 15%, and in some embodiments even between about 5% and about 15% anise oil (v/v).

### Cinnamon Leaf Oil (CAS 8007-80-05), Cinnamon Oil (CAS 8015-91-6)

Some embodiments of a composition as described herein comprise cinnamon leaf oil and/or cinnamon oil (comprising oil from the bark, fruit, leaf and/or twig).

Cinnamon is a spice obtained from the inner bark of several trees from the genus Cinnamomum. Species of cinnamon include Cinnamomum verum, C. burmannii, C. loureiroi, and C. aromaticum.

Cinnamon is considered to boost cognitive function, and memory, treat rheumatism, aid in digestion and relief of menstrual disorders. It is also considered as a cure for colds and fever, treatment for diarrhea, flatulence and other problems of the digestive system, treatment of toothache, bad breath, cough, headache, and heart disease, and has been suggested as a treatment for Alzheimer's disease and Type 2 diabetes. Cinnamon also serves as a natural food preservative, inhibiting bacterial growth and food spoilage.

### Clove Oil (CAS No. 8000-34-8)

Some embodiments of a composition as described herein comprise clove oil (comprising oil from the flower, bud and/or leaf).

Cloves (Syzygium aromaticum) are the aromatic dried flower buds of a tree in the family Myrtaceae.

Cloves are used in cooking, either in whole or in ground form, and as incense. Cloves are considered to be useful in relieving dental pain, and have been suggested as reducing blood sugar levels. Active compounds in cloves include eugenol, acetyl eugenol, beta-caryophyllene and vanillin; crategolic acid; tannins, gallotannic acid, methyl salicylate (painkiller); the flavonoids eugenin, kaempferol, rhamnetin, and eugenitin; triterpenoids like oleanolic acid, stigmasterol and campesterol; and several sesquiterpenes.

### Eucalyptus Oil 5

Eucalyptus is a diverse genus of flowering trees and shrubs in the myrtle family, Myrtaceae. Essential eucalyptus oils include citriodora (CAS No. 129828-24-6), radiata (CAS No. 92201-64-4), smithii (CAS No. 91771-68-5), staigeriana (CAS No. 91771-69-6) and globulus (CAS No. 8000-48-4).

Eucalyptus oil is used externally as an antiseptic; for treatment of burns, sores, ulcers, scrapes, boils and wounds. Eucalyptus oil is applied topically for relief of rheumatic pain, aches, stiffness and neuralgia. Inhalation of the vapor of boiled eucalyptus leaves is useful for relief of congestion, asthma and respiratory problems. Eucalyptus rubbed into the skin acts as an insect repellant, especially for mosquitoes and fleas. Rubbed into the chest, it relieves congestion and cough.

### Lavender Oil (Angustifolia CAS No. 8000-28-0; Latifolia CAS No. 8016-78-2)

Lavender is a genus of 39 species of flowering plants in the mint family, including Lavandula angustifolia, Lavandula latifolia, and Lavandula lanata.

Essential oil of lavender is used in balms, salves, perfumes, cosmetics, and topical applications, and is considered to have antiseptic and anti-inflammatory properties. Infusion of lavender is considered to soothe and heal insect bites and burns. Bunches of lavender repel insects. If applied to the temples, lavender oil soothes headaches. In pillows, lavender seeds and flowers aid sleep and relaxation.

Peppermint Oil (mentha piperita, CAS No. 8006-90-4)

Peppermint is a hybrid mint, a cross between the watermint (Mentha aquatic) and spearmint (Mentha spicata).

Medicinal uses of peppermint oil include uses in aiding digestion, reduction of motion sickness and upset stomach; as an antiseptic and breath freshener in dental care; relief of respiratory problems, including nasal congestion, sinusitis, asthma, bronchitis, cold and cough; relief of nausea and headache; relief of stress, depression and mental exhaustion; relief of irritable bowel syndrome; and pain relief. Peppermint oil is also considered to boost the immune system and improve blood circulation. Peppermint oil is used in hair care to remove dandruff and lice, and in skin care to nourish dull skin and improve oily skin.

### Isopropyl Alcohol (CAS No. 67-63-0)

Some embodiments of a composition as described herein comprise isopropyl alcohol as a solvent. Some embodiments comprise isopropyl alcohol together with at least one additional solvent. Some embodiments comprise isopropyl alcohol as a sole solvent.

It is believed that in some embodiments, the isopropyl alcohol helps wet the hair, assisting in providing a more even distribution of the composition over the hair and scalp, thereby assisting in ensuring that all lice are exposed to the composition.

Isopropyl alcohol, also known as rubbing alcohol, is considered relatively non-toxic and evaporates quickly. Long-term application to the skin can cause defatting. Isopropyl alcohol causes irritation of mucous membrane and eyes, and may cause dryness, irritation and dermatitis of the skin at high concentrations. In PCT patent publication WO 01/26471 is disclosed that isopropyl alcohol has an anti-lice effect and compositions disclosed thereon typically comprise between about 5% and 70% isopropyl alcohol.

In some embodiments, a composition comprises between about 10% and about 30%, in some embodiments between about 15% and about 25%, and in some embodiments even between about 15% and about 20% isopropyl alcohol (v/v). In some preferred embodiments, the composition comprises 20% (v/v) isopropyl alcohol.

### Isopropyl Myristate (CAS No. 110-27-0)

Some embodiments of a composition as described herein comprise isopropyl myristate as a solvent. Some embodiments comprise isopropyl myristate together with at least one additional solvent. Some embodiments comprise isopropyl myristate as a sole solvent.

Isopropyl myristate is the ester of isopropyl alcohol and myristic acid. Isopropyl myristate is known as having an anti-lice effect, which works by dissolving the wax that covers the exoskeleton of the lice, leading to water evaporation and death by dehydration. As noted above, Resultz® is a commercially-available composition for treating lice infestation including a combination of 50% isopropyl myristate and 50% silicon oil (cyclomethicone).

In some embodiments, a composition comprises between about 30% and about 75%, in some embodiments between about 30% and about 65%, in some embodiments between about 30% and about 50%, in some embodiments between about 30% and about 45%, and in some embodiments even between about 35% and about 45% isopropyl myristate (v/v). In some preferred embodiments, the composition comprises 40% (v/v) isopropyl myristate. In some preferred embodiments, the composition comprises between about 45% and about 50% (v/v) isopropyl myristate.

### Silicone Oil

Embodiments of compositions as described herein comprise a silicone oil.

Silicone oils (particularly dimethicone) are known for use in the treatment of head lice (Burgess, Ian F (2009) BMC Pharmacology 9(3):3, the commercially-available Resultz®), apparently acting as a lubricant, making it difficult for hatched lice to grasp hair and therefore easier to comb out. There is also evidence that silicone oils such as dimethicone have some (minor) a pediculicidal effect, blocking the breathing apparatus of lice, leading to suffocation.

In some embodiments, the silicone oil component of a composition as described herein acts as a lubricant, making it difficult for lice to grasp the hair, and additionally, making combing the hair simpler and less painful.

Any suitable silicone oil may be used in implementing a composition as described herein. Depending on the embodiment, suitable silicone oils include a polyalkyl siloxane, a polyaryl siloxane, a polyalkylaryl siloxane or a polyether siloxane copolymer, and combinations thereof. In some embodiments, the polyalkyl siloxane comprises polymethyl siloxane (dimethicone, e.g., CAS No. 9006-65-9).

Some embodiments of a composition as described herein comprise dimethicone as a silicone oil. Some embodiments comprise dimethicone together with at least one additional silicone oil. Some embodiments comprise dimethicone as a sole silicone oil.

In some embodiments, a composition comprises between about 5% and about 20%, in some embodiments between about 5% and about 15%, and in some embodiments even between about 5% and about 10% silicone oil (e.g., dimethicone) (v/v). In some preferred embodiments, the composition comprises 10% (v/v) silicone oil (e.g., dimethicone).

### Caprylic/Capric Triglyceride (e.g., CAS No. 73398-61-5)

Embodiments of the composition comprise an emollient ester of fatty acid derived from vegetable oils as an emollient and a diluent (helping reduce the chance of undesirable side effects). In some embodiments the emollient ester of fatty acid derived from vegetable oils as a lubricant making it difficult for lice to grasp the hair, and additionally, making combing the hair simpler and less painful.

Some embodiments of a composition as described herein comprise caprylic/capric triglyceride as an emollient ester of fatty acid derived from vegetable oils. Some embodiments comprise caprylic/capric triglyceride together with at least one additional emollient ester of fatty acid derived from vegetable oils. Some embodiments comprise caprylic/capric triglyceride as a sole emollient ester of fatty acid derived from vegetable oils.

Caprylic/capric triglyceride, also known as fractionated coconut oil, is commonly used as an emollient for calming and smoothing the skin. Caprylic/capric triglyceride is present in the commercially available anti-lice composition “Hairclean 1-2-3”® described in the introduction. In PCT patent publication WO 01/26471, an anti-lice composition is disclosed having between about 25% and 100% caprylic/capric triglyceride (Estasan GT 8-60).

In some embodiments, a composition comprises between about 5% and about 25%, in some embodiments between about 5% and about 20%, and in some embodiments even between about 10% and about 20% caprylic/capric triglyceride (v/v). In some preferred embodiments, the composition comprises 15% (v/v) caprylic/capric triglyceride.

Exemplary embodiments are discussed hereinbelow with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

### EXPERIMENTAL

### Example 1

### Exemplary Compositions

Exemplary compositions were prepared in accordance with the teachings herein using commercially-available ingredients.

The exemplary compositions prepared consisted of anise oil or rosemary oil as an essential oil; a combination of isopropyl myristate and isopropyl alcohol as solvent; and caprylic/capric triglyceride as emollient ester of fatty acid derived from vegetable oils. and dimethicone as a lubricant, in the concentrations shown in Table 1 below.

Isopropyl myristate and caprylic/capric triglyceride (Myritol 318) were purchased from Cognis (Monheim, Germany, a subsidiary of BASF SE, Ludwigshafen, Germany); isopropyl alcohol was purchased from Gadot Lab Supplies (Or Akiva, Israel); dimethicone was DC 1501 and/or DC350 both from Dow Corning Corporation (Midland, Mich., USA) and/or Belsil 1000 from Wacker Chemie AG (Munich, Germany).

TABLE 1
Exemplary compositions

| | Anise | Rosemary | Isopropyl | Isopropyl | | |
|---|---|---|---|---|---|---|
| Composition | oil | oil | alcohol | myristate | Dimethicone | Caprylic/ |
| designation | (% v/v) | (% v/v) | (% v/v) | (% v/v) | (% v/v) | capric TG (% v/v) |
| 002 | — | 15 | 20 | 40 | 10 | 15 |
| 004 | 15 | — | 20 | 40 | 10 | 15 |
| 005 | — | 10 | 20 | 40 | 10 | 20 |
| 006 | — | 5 | 20 | 40 | 10 | 25 |
| Paranise | 5 | — | 20 | 48 | 10 (DC350) | 17 |
| Rosinix | — | 5 | 20 | 48 | 10 (DC350) | 17 |
| control | — | — | — | | — | 100 |

### Example 2

### Preparation of an Exemplary Composition

The components were added at room temperature to a mixer equipped with an agitating planetary propeller and a high-shear homogenizer or dispersing disc and mixed until a clear solution was achieved, after about 5 minutes.

### Example 3

### Pediculicidal Activity of the Exemplary Compositions

The pediculicidal and ovicidal activities of several exemplary compositions were tested in the laboratory on the human body louse according to the following procedures:

Body lice (Pediculus humanus humanus) were reared in the laboratory according to the method described by Cole M. M. [Body lice. In: Insect colonization and mass production. Smith, C. N. (ed.), Academic Press, New York, N.Y., USA, p. 15-24 (1966)]. For each test, 50 lice (10 males, 10 females and 30 nymphs) were placed on a 7-cm white filter paper disc and exposed to 1 g of an exemplary composition. The lice were left in contact with the composition for 5-15 min. The lice were then removed from the filter paper, washed with normal shampoo (1:20) for 1 min and then with tap water for 1 min. After treatment, lice were transferred to a fresh filter paper disc and incubated overnight at optimum temperature and humidity. Mortality was determined after 24 hrs. As a negative control, lice treated with 40% ethyl alcohol, were used. Each exposure time was tested in triplicate.

### Example 4

### Ovicidal Activity of the Exemplary Compositions

Lice were placed on human hair every other day for 5 days and left for 24-48 hrs for oviposition. Fifty eggs, 2-6 days old were treated with the exemplary compositions, as described above. The number of hatched and non-hatched eggs was counted after 10 days.

The results of the tests are presented in Table 2 below.

TABLE 2
Pediculicidal and ovicidal activity of exemplary compositions

| | | Mean % | Mean % |
|---|---|---|---|
| Composition | Exposure | mortality | mortality in |
| designation | time (min) | in lice | eggs |
| 002 | 5 | 100 | 100 |
| | 10 | 100 | 100 |
| | 15 | 100 | 100 |
| 004 | 5 | 100 | 98.3 |
| | 10 | 100 | 100 |
| | 15 | 100 | 100 |
| 005 | 5 | 100 | 98.3 |
| | 10 | 100 | 100 |
| | 15 | 100 | 100 |
| 006 | 5 | 100 | 98.3 |
| | 10 | 100 | 99.3 |
| | 15 | 100 | 100 |
| Paranise | 5 | 100 | 100 |
| | 10 | 100 | 100 |
| | 15 | 100 | 100 |
| Rosinix | 5 | 100 | 98.3 |
| | 10 | 100 | 99.3 |
| | 15 | 100 | 100 |
| Control | 15 | 12 | 16 |

### Example 5

### Clinical Study of Exemplary Compositions

For each one of compositions Paranise and Rosinix, 30 human children aged 5 to 15 years (average age: 8.8 years, 82% females, 75% having long hair) were treated with a single application of the respective composition to study the clinical efficacy thereof.

### Before Application of a Composition

The hair of the children was examined by a qualified nurse. It was ascertained that the hair of ˜90% of the children was infested with lice (mean: ˜30 lice) and nits (mean: ˜150 nits). ˜15% of the children had greater than 200 lice.

### Application of Composition

A child's hair was dampened. The respective composition (Paranise or Rosinix) was applied to the damp hair of the child, and massaged-in to ensure that the composition contacted substantially all the hair and scalp. A qualified nurse combed a section of the hair with a louse-comb in the usual way at 5, 10, 15 and 20 minutes after application of the composition. After the combing subsequent to 20 minutes from application of the composition, the child's hair was rinsed with water and the qualified nurse thoroughly combed the hair with a louse-comb in the usual way. After another 7 days, a qualified nurse thoroughly combed the child's hair with a louse-comb in the usual way.

Five Minutes after Application of Composition

Five minutes after application of a composition, a first section of the child's hair was combed with a louse-comb. In ˜80% of the children, only dead lice and no living lice were recovered from the louse-comb.

Ten Minutes after Application of a Composition

Ten minutes after application of a composition, a second section of the hair (different from the first section) of the child's hair was combed with a louse-comb. In all of the children, only dead lice and no living lice were recovered from the louse-comb.

Fifteen and Twenty Minutes after Application of a Composition

Fifteen and twenty minutes after application of a composition, no living lice were recovered using the louse comb from the hair of any of the children.

### After Rinsing and Brushing

After the composition was rinsed from a child's hair, the hair was brushed and combed using a louse-comb in the usual way. No substantial residue of the composition was observed in the hair of any of the children. Subsequently, the hair was thoroughly combed with a louse-comb. No dead lice were recovered from the louse-comb of ˜80% of the children, and an average of 2 dead lice were recovered from the louse comb of ˜20% of the children.

### After Seven Days

Seven days after the treatment, a few nits were found in the hair of some of the children. In the hair of two of the children who previously had more than 200 living lice, a few living nymphs were found.

### CONCLUSIONS

The composition Paranise and Rosinix were well-tolerated (including acceptable scent), easy to apply and no adverse reactions were observed. After rinsing of the hair, no substantial residue of compositions was noted in the hair.

The compositions were found to be highly effective in treating lice infestation even at short application times. The presence of several living nymphs in some children indicate that it may be advisable to repeat treatment after about 7 days in cases of exceptionally serious infestation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the invention.

Section headings are used herein to ease understanding of the specification and should not be construed as necessarily limiting.

What is claimed is:

1. A composition comprising:
an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof;
from about 10% to about 30% (v/v) isopropyl alcohol;
from about 30% to about 50% (v/v) isopropyl myristate;
between about 5% and about 20% (v/v) of a silicone oil; and
from about 5% to about 25% (v/v) capric/caprylic trigylceride.

2. The composition of claim 1, wherein said essential oil is present at a concentration of from about 1% to about 20% (v/v).

3. The composition of claim 1, wherein said essential oil is selected from the group consisting of anise oil and rosemary oil.

4. The composition of claim 1, wherein said isopropyl alcohol is present at a concentration of between about 15% and about 25% (v/v).

5. The composition of claim 1, wherein said isopropyl alcohol is present at a concentration of between about 15% and about 20% (v/v).

6. The composition of claim 1, wherein said isopropyl myristate is present at a concentration of between about 30% and about 45% (v/v).

7. The composition of claim 1, wherein said isopropyl myristate is present at a concentration of between about 35% and about 45% (v/v).

8. The composition of claim 1, wherein said silicone oil comprises dimethicone.

9. The composition of claim 1, wherein said silicone oil is present at a concentration of between about 5% and about 15% (v/v).

10. The composition of claim 1, wherein said silicone oil is present at a concentration of between about 5% and about 10% (v/v).

11. The composition of claim 1, wherein said capric/caprylic trigylceride is present at a concentration of between about 5% to about 20% (v/v).

12. The composition of claim 1, wherein said capric/caprylic trigylceride is present at a concentration of between about 10% and about 20% (v/v).

13. The composition of claim 1, the composition formulated for topical administration.

14. The composition of claim 1, wherein:
said essential oil is selected from the group consisting of anise oil and rosemary oil at a concentration of from about 1% to about 20% (v/v);
between about 15% and about 25% (v/v) isopropyl alcohol;
between about 30% and about 50% (v/v) isopropyl myristate;
from about 5% to about 20% (v/v) of dimethicone; and
from about 5% to about 25% (v/v) capric/caprylic trigylceride.

15. The composition of claim 14, comprising one of rosemary oil and anise oil as sole essential oil.

16. The composition of claim 14, wherein said isopropyl alcohol is present at a concentration of between about 15% and about 20% (v/v).

17. The composition of claim 14, wherein said isopropyl myristate is present at a concentration of between about 30% to about 45% (v/v).

18. The composition of claim 14, wherein said isopropyl myristate is present at a concentration of between about 35% and about 45% (v/v).

19. A method for the treatment of pediculosis, comprising topically administering to a subject in need thereof a composition comprising:
an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof;
from about 10% to about 30% (v/v) isopropyl alcohol;
from about 30% to about 50% (v/v) isopropyl myristate;
between about 5% and about 20% (v/v) of a silicone oil; and
from about 5% to about 25% (v/v) capric/caprylic trigylceride.

20. A method for the preparation of a composition useful in the treatment of peidculosis comprising mixing together:
an essential oil selected from the group consisting of anise oil, rosemary oil, calendula oil, tea tree oil, sassafras oil, quassia oil, cinnamon oil, clove oil, eucalyptus oil, lavender oil, peppermint oil, or combinations thereof;
from about 10% to about 30% (v/v) isopropyl alcohol;
from about 30% to about 50% (v/v) isopropyl myristate;
between about 5% and about 20% (v/v) of a silicone oil; and
from about 5% to about 25% (v/v) capric/caprylic trigylceride.
